(19) United States
(12) Patent Application Publication
(10) Pub. No.: US 2022/0331114 A1
(43) Pub. Date: Oct. 20, 2022

(54) IMPLANT FOR THE GLENO-HUMERAL JOINT

(71) Applicants: Marco MAIOTTI, ROMA (IT); Raffaele RUSSO, NAPOLI (IT)

(72) Inventors: Marco MAIOTTI, ROMA (IT); Raffaele RUSSO, NAPOLI (IT)

(21) Appl. No.: 17/811,002

(22) Filed: Jul. 6, 2022

Related U.S. Application Data
(63) Continuation-in-part of application No. 16/969,391, filed on Aug. 12, 2020, now abandoned.

(30) Foreign Application Priority Data
Feb. 16, 2018 (IT) 102018000002734

(51) Int. Cl. A61F 2/30; A61F 2/40

(52) U.S. Cl. CPC A61F 2/30734; A61F 2/4081; A61F 2002/30736; A61F 2002/30266; A61F 2310/00359; A61F 2002/30461; A61F 2002/30192; A61F 2002/30784; A61F 2002/30836; A61F 2002/30909; A61F 2310/00023; A61F 2310/00131

(57) ABSTRACT

The present invention relates to an implant (1) for the correction of gleno-humeral instability, in particular for the correction of a glenoid defect (Dg) of the glenoid (G) of a patient, said implant (1) having a substantially flat portion (3), said substantially flat portion (3) being suitable, when said implant (1) is in use, for being placed at the site of the glenoid defect (Dg) in contact with the external bone portion of the glenoid (G) of a patient, said implant (1) being characterized in that at least said flat portion (3) is made of heterologous deproteinized bone material.

### CROSS-REFERENCE TO RELATED APPLICATION

This application is a continuation-in-part patent application of the U.S. utility patent application Ser. No. 16/969,391 filed Aug. 12, 2020, which was a United States National Stage Application filed under 35 U.S.C 371 of PCT Patent Application No. PCT/IT2019/050029, filed Feb. 13, 2019, which claims ITALIAN Patent Application Serial No. IT 102018000002734, filed Feb. 16, 2018, the disclosure of all of which are hereby incorporated by reference in their entirety.

### SUMMARY OF THE INVENTION

The present invention relates to an implant for the gleno-humeral or shoulder joint.

More precisely, the present invention relates to an implant for the correction of gleno-humeral instability.

The gleno-humeral or shoulder joint is the point of articulation between the glenoid cavity of the scapula and the head of the humerus, which allows a wide range of movements of the upper limb.

The physiognomy that allows this wide range of movements causes a relative instability of the joint.

In its chronic form, gleno-humeral instability represents a pathology that is difficult to understand and solve. The majority of diagnostic studies and anatomopathological surveys have demonstrated that there exists a bone defect of the glenoid in over 80% of the cases of chronic anterior instability.

In order to definitively solve the problem, the surgeon must undertake to correct the glenoid defect with a bone graft. Bone grafts can be homologous, autologous and heterologous. They can be harvested from structures neighbouring the shoulder or at a distance.

All of the scientific literature is in agreement as to the necessity of repairing this defect in the most anatomical way possible, but this particular aim cannot always be achieved without altering the patient's anatomy.

If a piece of autologous bone is used, a second surgical intervention in the same patient will be necessary in order to harvest it.

If an allograft from a donor is used, it will be necessary to rely on bone banks, with all the problems tied to this type of procurement.

In both the former and latter cases, the operating time will be increased and the graft will never be standardized in its form, because it must be cut freehand by the surgeon at the time of the intervention.

In the case of a heterologous bone graft, there are no available data in this regard for application on the glenoid for anterior or posterior instability, which the scientific literature is very critical toward. Even for autologous grafts, taken from neighbouring structures or from the iliac crest, there are no certainties with respect to osteointegration, especially if they are composed of only bone and thus lacking a vascular peduncle. In fact, in all of the cases already experimented with and applied in shoulder surgery, the transfer of bone from the iliac crest takes place freely, that is, without a vascular peduncle grafted on another vessel to enable a direct supply of the transplanted piece of bone.

To date, no prosthetic solutions for the correction of the defect itself have been described. The problems are tied to the survival of heterologous tissues and the antigenic reaction of the host macrophages, which would destroy the active antigenic parts present on the graft. In the case of mineral scaffolds, the difficulty up to now has been to create the means of stability.

The aim of the present invention is to develop an implant to correct the glenoid defect, which allows the problems of the prior art to be solved by means of a solution that may be optimally adapted to the patient's anatomy.

Furthermore, the aim of the present invention is to provide an implant that may be easily and quickly produced, so as to be available in shorter times and at reduced costs.

In addition, the aim of the present invention is to obtain an implant that is quickly and effectively osteointegrated, for a faster recovery of the patient.

The object of the present invention is an implant for the correction of gleno-humeral instability, in particular for the correction of the glenoid defect of the glenoid of a patient, said implant having a substantially “J” shape with a wedge portion substantially transversal to a substantially flat portion, said wedge portion being suitable for being inserted into the bone channel obtained in the glenoid of a patient, said substantially flat portion being suitable, when said implant is in use, for being placed at the site of the glenoid defect in contact with the external bone portion of the glenoid of a patient, said implant being characterized in that it comprises a first element made of organic material corresponding at least to the flat portion of the implant, and a second element made of a rigid biocompatible material corresponding to at least the wedge portion of the implant and coupled to said first element.

In particular, according to the invention, the organic material of the first element can be a biocompatible material which is suitable for being inhabited by bone cells from the glenoid bone, so as to facilitate the stabilization of the implant.

Again according to the invention, the organic material of the first element can be a biological or mineral material; in particular it is a bone material, preferably a heterologous deproteinized bone material.

Likewise according to the invention, said second element can be made of a porous or non-porous biocompatible material, or biocompatible metal such as titanium or tantalum.

Furthermore, according to the invention, said second element can have at least one rough surface in the wedge portion of the implant, so as to facilitate the grasp of the implant on the bone channel.

Moreover, according to the invention, the first element can have a “J” shape having a flat portion and a wedge portion substantially transversal to said flat portion.

According to the invention, said second element can preferably be a sheath that, when coupled to the first element, at least partially overlaps the wedge portion of the first element.

Again according to the invention, said sheath can have a mesh or a grid that forms said rough surface.

Likewise according to the invention, the first element can correspond to the flat portion of the implant, the second element can correspond to the wedge portion of the implant, wherein the second element can be coupled to the first element by means of coupling means.

Furthermore, according to the invention, the coupling means can be pins present in said second element and suitable for being inserted in respective holes obtained in the first element.

Again according to the invention, the second element can have a plurality of holes making up the rough surface of the second element.

Preferably, according to the invention, the rough surface in the wedge portion of the implant can be obtained by knurling.

In particular, according to the invention, the wedge portion of the implant can be substantially curved to ensure a more effective grasp on the bone channel wherein it is inserted.

Furthermore, according to the invention, said implant can have a first hole in the flat portion, suitable for allowing the passage of a stabilization thread for the stabilization of the implant.

Finally, according to the invention, said implant can have a second hole in the wedge portion suitable for allowing the passage of a traction thread to facilitate the insertion of the implant into the glenoid channel.

It is further object of the present invention an implant for the correction of gleno-humeral instability, in particular for the correction of a glenoid defect of the glenoid of a patient, said implant, having a substantially flat portion, said substantially flat portion being suitable, when said implant is in use, for being placed at the site of the glenoid defect in contact with the external bone portion of the glenoid of a patient, said implant being characterized in that at least said flat portion is made of heterologous deproteinized bone material.

In particular, according to the invention, said implant may have at least a first hole in the flat portion suitable for allowing the passage of at least a thread or strand for the stabilization of the implant, preferably it has two holes.

More in particular, according to the invention, the cortical part of the bone may be arranged in correspondence of a first outer side of the flat portion of the implant and the spongier part of the bone may be arranged in correspondence of a second inner side of the flat portion, opposite to the first side.

Always according to the invention, said implant may have at least a first through hole passing through said first outer side and send second inner side of the flat portion.

Again according to the invention, said flat portion may have a plurality of holes suitable for allowing the insertion of stabilization means for facilitating the stabilization of the implant in the glenoid.

Further according to the invention, said implant may have a substantially J-shape with a wedge portion substantially transversal to said substantially flat portion, said wedge portion being suitable for being inserted into the bone channel obtained in the glenoid of a patient, in that said wedge portion is made of a biocompatible rigid material is coupled to said flat portion.

Additionally according to the invention, said flat portion and said wedge portion may be integral with each other and may be made of heterologous deproteinized bone material.

In particular, according to the invention, said implant may have at least a second hole obtained in the wedge portion suitable for allowing the passage of at least a thread or strand.

More in particular, according to the invention, the wedge portion may be coupled to the flat portion by means of coupling means.

Further according to the invention, said wedge portion may be made of a porous or non-porous biocompatible material, or a biocompatible metal such as titanium or tantalum.

Again according to the invention, said flat portion may provide a plurality of holes, in that the coupling means are a plurality of pins in said wedge portion suitable for being inserted in the respective holes of said flat portion.

Always according to the invention, the wedge portion may have a plurality of holes that make up a rough surface, so as to facilitate the grasp of the implant on the bone channel.

Preferably according to the invention, the wedge portion of the implant may have a rough surface obtained by knurling, so as to facilitate the grasp of the implant on the bone channel.

Again according to the invention, the wedge portion of the implant may be substantially curved so as to more effectively grasp the bone channel wherein it is inserted.

It is further object of the present invention a kit comprising the implant described, and stabilization means for facilitating the stabilization of the implant in the glenoid.

In particular, according to the invention, said stabilization means may comprise at least a thread or strand.

More in particular, according to the invention, said stabilization means may further comprise at least one button suitable for coupling with said at least one thread.

It is also object of the present invention a kit comprising the implant described comprising a wedge portion, and stabilization means for facilitating the stabilization of the implant in the glenoid, wherein said stabilization means comprise at least a second element, said second element being a sheath that, when it is coupled to the implant, at least partially overlaps the wedge portion.

Finally, according to the invention, said sheath may have a mesh or grid that forms a rough surface, so as to facilitate the grasp of the implant on the bone channel.

### DESCRIPTION OF DRAWINGS

The invention will now be described by way of illustration and not by way of limitation, with particular reference to the drawings of the appended figures, wherein:

FIG. 1a shows a perspective view of the implant according to the invention in a first embodiment;

FIG. 1b shows an exploded perspective view of the implant of FIG. 1a;

FIG. 2a shows a perspective view of the implant according to the invention in a second embodiment;

FIG. 2b shows an exploded perspective view of the implant of FIG. 2a;

FIG. 3a shows a perspective view of the implant according to the invention in a third embodiment;

FIG. 3b shows an exploded perspective view of the implant of FIG. 3a;

FIG. 3c shows an exploded top view of the implant of FIG. 3a;

FIG. 3d shows a side view of the implant of FIG. 3a;

FIG. 4a shows a perspective view of the implant according to the invention in a fourth embodiment;

FIG. 4b shows a side view of the implant of FIG. 4a;

FIG. 4c shows a top view of the implant of FIG. 4a;

FIG. 5a shows a perspective view of the implant according to the invention in a fifth embodiment;

FIG. 5b shows a side view of the implant of FIG. 5a;

FIG. 6a shows a perspective view of the implant according to the invention in a sixth embodiment;

FIG. 6b shows an exploded perspective view of the implant of FIG. 6a;

FIG. 7a shows a top view of the implant according to FIG. 6a during the phase of application at the site of the defect of a glenoid;

FIG. 7b shows a perspective view of FIG. 7a;

FIG. 8a shows a top view of the implant according to FIG. 6a after the application thereof for the correction of the glenoid defect of FIG. 7a;

FIG. 8b shows a perspective view of FIG. 8a;

FIGS. 9a-9c show sectional side views of the steps of applying the implant of FIG. 6a for the correction of the defect of a glenoid, highlighting the internal structure of the bone component;

FIG. 10 shows a perspective view of the implant according to the invention in a seventh embodiment;

FIG. 11a shows a side cross-section view of a glenoid;

FIG. 11b shows a side cross-section view of the glenoid of FIG. 11a where the implant according to FIG. 10 is applied in correspondence of the glenoid defect;

FIG. 11c shows a cross-section along section plane XI of the implant applied to the defect of FIG. 11b;

FIG. 12 shows a perspective view of the implant according to the invention in a eighth embodiment;

FIG. 13a shows a top perspective view of a kit according to the invention comprising the implant according to FIG. 12;

FIG. 13b shows a rear perspective view of the kit of FIG. 13a;

FIGS. 13c and 13d show respectively two side perspective views of the kit according to FIG. 13a applied to a glenoid;

FIG. 14 shows a perspective view of the implant according to the invention in a ninth embodiment;

FIG. 15a shows a side cross-section view of a glenoid;

FIG. 15b shows a side cross-section view of the glenoid of FIG. 15a where the implant according to FIG. 14 is applied in correspondence of the glenoid defect; and

FIG. 15c shows a cross-section along section plane XV of the implant applied to the defect of FIG. 15b.

### DETAILED DESCRIPTION

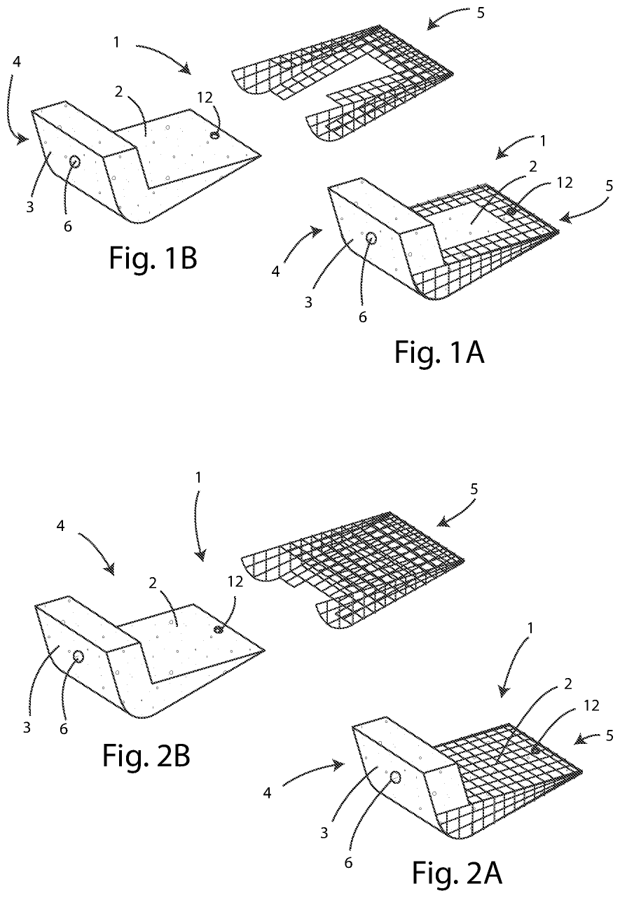

Making reference to FIGS. 1-6, different embodiments of the implant according to the present invention for the correction of a glenoid defect in the gleno-humeral joint, indicated by the reference number 1 are shown.

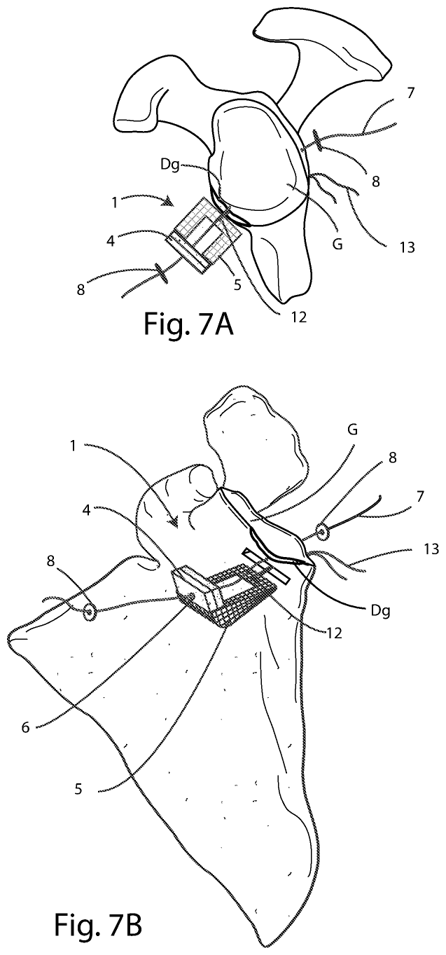

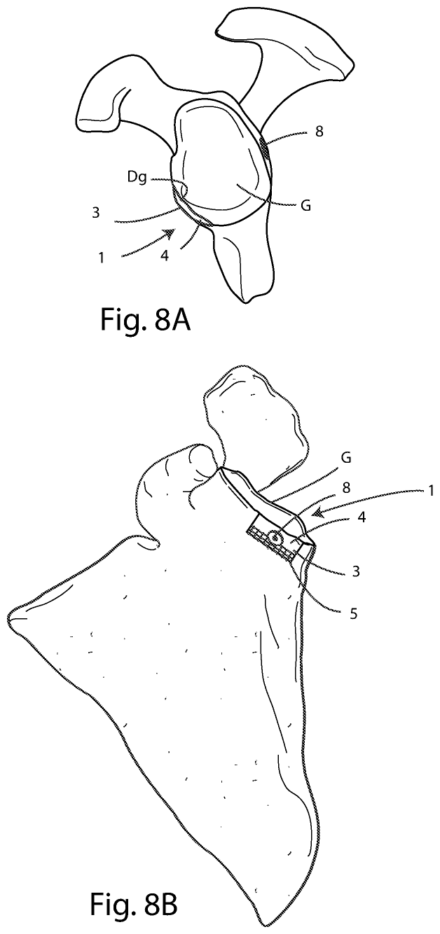

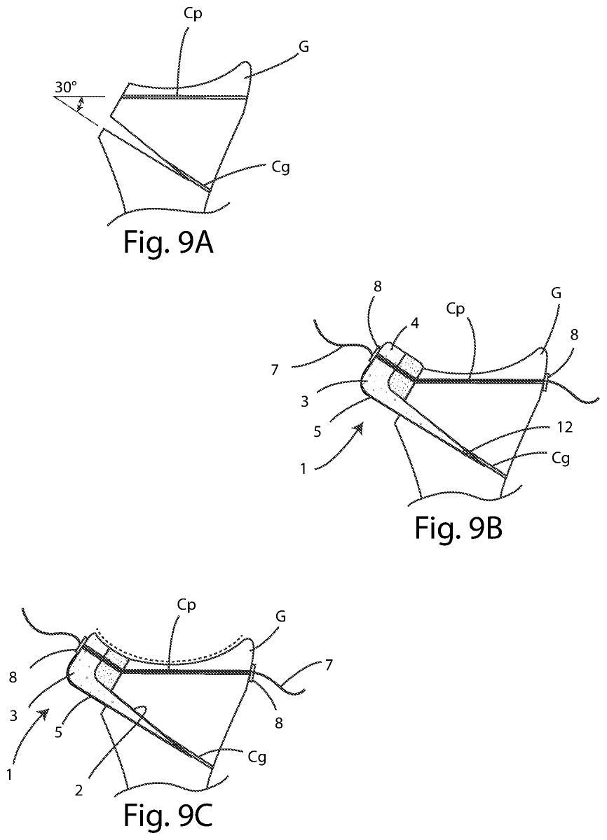

Said implant 1 has a “J” shape having a wedge portion 2 transversal to a substantially flat portion 3. As shown, for example, in FIGS. 7-9, the wedge portion 2 is suitable for being inserted in the bone channel Cg previously obtained in the glenoid G being operated on; whereas the substantially flat portion 3 is suitable, when the implant 1 is in use, for being placed at the site of the glenoid defect Dg in contact with the external bone portion of the glenoid G of the patient being operated on.

Furthermore, said implant 1 comprises a first element 4 made of organic material at least in the flat portion 3 of the implant 1.

For organic material, reference can be made to a biological or mineral material, such as, for example, bone material, for example heterologous deproteinized bone material or any other biocompatible material that can be inhabited by bone cells from the glenoid bone, so as to facilitate the stabilization of the implant 1.

Advantageously the fact that the implant 1 according to the invention is made of heterologous deprotenized bone as compared to homologous bone provides the following benefits:
the block can be cut in shape with precise machining apparatuses and thus can be industrialized, while the homologous bone has to be cut by the surgeon during the surgical intervention and thus the shape obtained is not very precise;
furthermore it is possible to obtain a plurality of different shapes of the block adapted for the different kinds of defects of the glenoid bone, thus specific for each patient or category of patients to restore better the anatomy;
after years from the intervention, the homologous bone block according the prior art ostoeintegrates too much with the patient's glenoid bone and thus its shape is deformed not being able to cover the bone loss, the heterologous deprotenized bone block according to the invention acts as a prosthesis and even after years from the intervention its shape is maintained almost unchanged.

Said bone portion is in particular of a heterologous type, and can be previously profiled and standardized in shape and size. For example, the preferred sizes can be a length of 2 cm, width of 8 cm and thickness of 15 mm.

As shown in FIGS. 6-9, the part represented with a higher density of dots in the bone component corresponds to the spongier part of the mineral component of the heterologous bone, and the part represented with a lower density of dots is the cortical part of the bone originating from the animal and thus the more compact part.

Furthermore, the implant 1 provides for a second element 5 made of rigid biocompatible material and coupled to said first element 4. Said second element 5 being preferably made of rigid biocompatible material, for example pyrocarbon, or tantalum or another porous or non-porous biocompatible material, or biocompatible metal such as titanium or tantalum, so as to improve the possibility of stabilization, synthesis and osteointegration of the implant 1 with the glenoid being operated on.

In particular, this enables greater stability and the possibility for the first element 4, in particular the bone component thereof, to have time to adapt itself.

Said second element 5 has at least one rough surface in the wedge portion 2 of the implant 1, which facilitates the grasp of the implant 1 on the bone channel Cg.

As shown in the figures, the implant 1 has holes 6, 12, a first hole 6 obtained in the flat portion 3 and a second hole 12 obtained in the wedge portion 2.

As shown in the FIGS. 7a-7b, 8a-8b and 9a-9c, the first hole 6 acts as the second stabilization point, since it enables the passage of a thread 7. According to the invention is provided a kit comprising the implant 1 according to the invention, one or more threads 7, 13 and one or more buttons 2. The use of two buttons 8 which are inserted at the two ends of the thread 7 or stabilization thread can be provided for, one 8 abutting on the flat portion 2 of the implant 1 and one 8 abutting on an external wall of the glenoid bone G, so as to facilitate the stabilization of the implant 1 and compact the tissue on the glenoid neck.

The second hole 12 allows the passage of a further thread or traction thread 13 necessary for the insertion of the implant 1 at the site of the glenoid defect to be repaired; it thus serves temporarily to draw, once passed posteriorly to the scapula, the flat portion 2 into the opening Cg for the penetration thereof into the glenoid bone G.

The bone channel Cg obtained in the glenoid bone G is preferably oriented by 30° relative to the passage channel Cp of the traction thread 13. The bone channel Cg preferably has a diameter equal to 2.8 mm.

The process for the insertion of the implant according to the invention provides for the following steps:

a) use of a cannula with a dedicated shape, preferably with a 16 mm diameter, to be inserted through the rotator interval;

b) use of a pin with a rear eye for the passage of a medial guide thread;

c) use of a guide for the hole Cp parallel to the glenoid surface for the passage and stabilization of the implant 1 with the button 8;

d) use of a dedicated curved scalpel with a thin wedge shape at the start of the blade, then increasing in thickness, to fashion the glenoid opening at an angle of about 100° relative to the glenoid surface;

e) insertion of the implant 1 in the glenoid opening fashioned,

f) stabilization of the rigid element of the implant by pulling in the slot by the guide thread 13, which is oblique by 100°, and by the rough wedge 2 itself, which becomes implanted in the slot Cg fashioned in the glenoid cavity G with the suitably designed curved scalpel, and by the button 8 with non-resorbable thread 7, which passes in the tunnel Cp parallel to the glenoid surface made with the rear guide and rear button 8,

g) once inserted into the glenoid slot, the implant is impacted with a special curved tamp and is definitively stabilized by tying the thread 7 on it with a dynamometer.

In FIGS. 1a and 1b, a first and preferred embodiment of the implant 1 according to the invention is shown, wherein it is shown a kit comprising a first element 4 corresponding to the implant 1, which is completely made of organic material, in particular bone material, in particular of a heterologous type, and has a “J” shape as described previously, thus with a substantially flat portion 3 that is substantially transversal to a wedge portion 2. The kit further comprises a second element 5 which corresponds to a sheath made of metal material that, when it is coupled to the first element 4, at least partially overlaps the wedge portion 2 of the first element 4 so as to protect it and decrease the risk of breakage during implantation in the bone channel Cg.

The sheath 5 consists of a mesh or metal grid that thus has a rough surface which improves the adhesion of the wedge portion 2 with the bone channel Cg into which the implant 1 is inserted.

In FIGS. 2a-2b, an alternative embodiment to the one in FIG. 1 is shown, which differs in that the second element 5 completely covers the wedge portion 2 of the first element 4.

As shown for the embodiment in FIGS. 6a-6b, the sheath 5 can optionally also cover part of the flat portion 2 of the first element 4, up to the first hole 6.

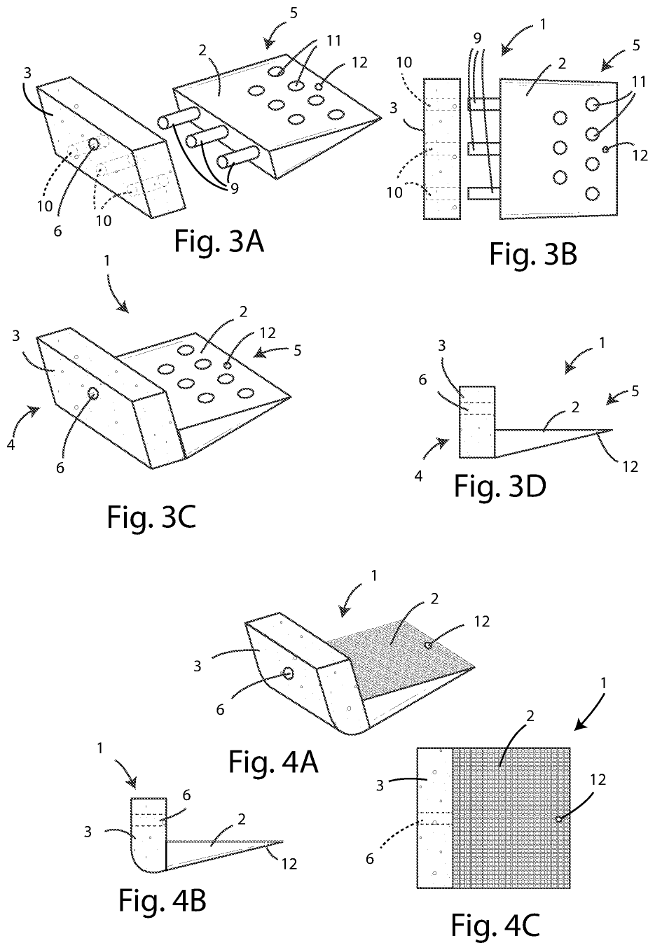

In reference to FIGS. 3a-3d, they show a third embodiment of the implant 1 according to the invention, wherein the first element 4 is made of an organic material, in particular a bone material, preferably heterologous, and corresponds to the flat portion 3 of the implant 1, whereas the second element 5 is made of a biocompatible material, preferably metal, and corresponds to the wedge portion 2 of the implant 1 and is coupled to the first element 4 by means of pins 9 suitable for being inserted in respective holes 10 obtained in the first element 4.

Furthermore, the second element 5 has holes 11 that make up the rough surface of the second element 5.

In FIGS. 4a-4c, a fourth embodiment of the implant 1 according to the invention is shown, which differs from the one in FIGS. 3a-3d in that the rough surface in the wedge portion 2 of the implant 1 is obtained by knurling.

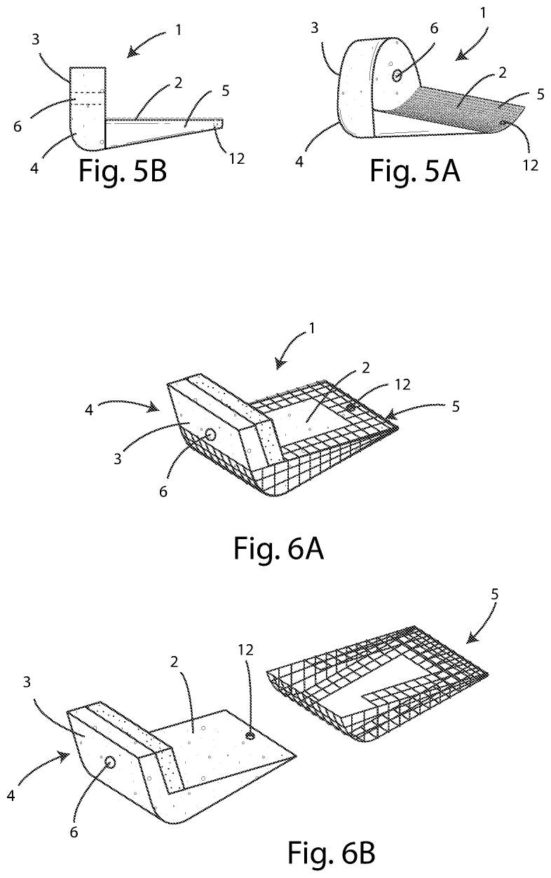

The implant 1 according to the embodiment in FIGS. 5a and 5b differs from the one in FIGS. 4a-4c in that it has the wedge portion 2 curved so as to more effectively grasp the bone channel C_{g} wherein it is inserted. More specifically, said wedge portion 2 has an axis of curvature parallel to the length of the wedge portion 2.

This geometry can also be provided for the other embodiments described.

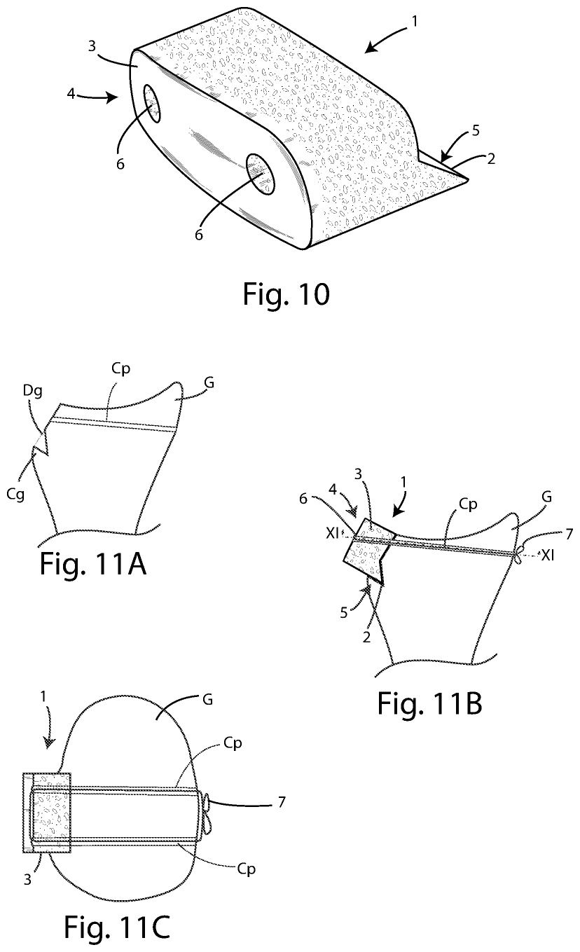

With reference to FIGS. 10 and 11a-11c it is shown a seventh embodiment of the implant 1 according to the invention. Said implant 1 has a “J” shape having a wedge portion 2 transversal to a substantially flat portion 3. Just like in the first embodiment according to FIG. 1, the the wedge portion 2 is suitable for being inserted in the bone channel Cg previously obtained in the glenoid G being operated on; whereas the substantially flat portion 3 is suitable, when the implant 1 is in use, for being placed at the site of the glenoid defect Dg in contact with the external bone portion of the glenoid G of the patient being operated on.

Said implant 1 comprises a flat portion 3 and a wedge portion 2 which are integral among each other and are made of organic material as described above, in particular a biocompatible rigid material, preferably heterologous deproteinized bone material.

More in particular, the cortical part of the bone (shown by means of parallel lines) is arranged in correspondence of the outer flat side of the flat portion 3, in particular in correspondence of the side opposite to the side where is arranged the wedge portion 2, while the spongier part of the bone (shown by means of dots) is arranged in correspondence of the inner side of the flat portion 2 and in correspondence of the wedge portion 3.

Furthermore, the implant 1 has two through holes 6 obtained in the flat portion 3, substantially transversal to the outer and inner sides of the flat portion 3 for the passage of stabilization means, such as threads 7 or strands.

As shown in FIGS. 11a-11c it is shown a kit according to the invention, comprising the implant 1 described, and at least one thread 7. The implant 1 is coupled and fixed to the glenoid bone G in correspondence of the defect. In particular the wedge portion 2 acts as a first stabilization means, stabilizing the position of the implant 1 to the glenoid bone G, subsequently threads 7 or strands are inserted along the holes 6 and the channels Cg obtained in the glenoid bone G in order to fix and stabilize the implant 1 to the glenoid bone G. In particular, the threads or strands 7 are then knotted on one or both sides of the glenoid bone G.

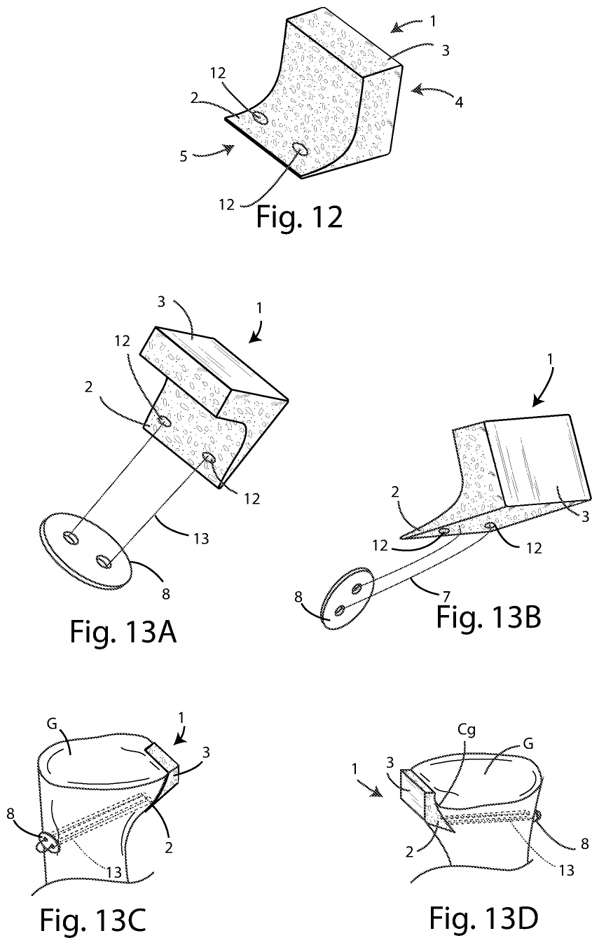

With reference to FIGS. 12 and 13a-13d it is shown an eighth embodiment of the implant 1 according to the invention.

Said eighth embodiment differs from the seventh embodiment described above in that it is devoid of holes in correspondence of the flat portion 3, but comprises two through holes 12 in correspondence of the wedge portion 2. In particular, the two holes 12 are obtained along an axis perpendicular to the top and bottom sides of the wedge portion 2.

In FIGS. 13a and 13b is shown a kit according to the invention comprising the implant 1 described, a thread 13 and a button 8 having two holes. Said kit is similar to the kit described for the embodiments of FIGS. 1-9 as it provides inserting the implant 1 in correspondence of the bone glenoid defect Dg with the flat portion 3 in contact with the glenoid bone G and the wedge portion 3 inserted in the bone channel Cg obtained previously on the glenoid bone G. The thread 13 is inserted through the holes 12 in the wedge portion 3 of the implant and passes through the channels Cg obtained in the glenoid bone G and is afterwards tied passing through the holes on the button 8 abutting against the other side of the glenoid bone G, as shown in FIGS. 13c and 13d.

In other embodiments according to the invention, said implant 1 might provide holes both in in correspondence of the wedge portion 2 as in the flat portion 3.

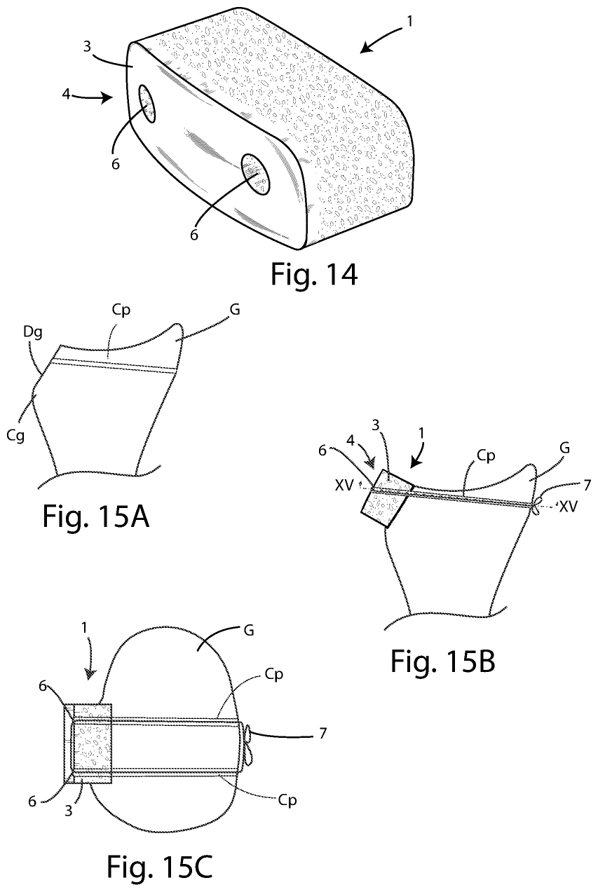

Making reference to FIGS. 14 and 15a-15c it is shown a ninth embodiment of the implant 1 according to the invention.

Such embodiment is very similar to the embodiment of FIG. 10, however it does not provide the wedge portion 2.

The implant 1 provides a flat portion 3 made of organic material as described above, in particular a biocompatible rigid material, preferably heterologous deproteinized bone material.

More in particular, the cortical part of the bone (shown by means of parallel lines) is arranged in correspondence of the outer flat side of the flat portion 3, while the spongier part of the bone (shown by means of dots) is arranged in correspondence of the side of the flat portion 2 opposite to the outer side. Such inner side is the side that will be placed in correspondence of the glenoid bone defect.

Furthermore, the implant 1 has two through holes 6 obtained in the flat portion 3, substantially transversal to the outer and inner sides of the flat portion 3 for the passage of stabilization means, such as threads-7 or strands.

Preferably the preferred size of said implant 1 is 22 mm in length, 10 mm in height, and 9 mm in thickness.

In FIGS. 15a-15c it is shown a kit according to the invention, comprising the implant 1 described, and at least one thread 7. The implant 1 is coupled and fixed to the glenoid bone G in correspondence of the defect. In particular the wedge portion 2 acts as a first stabilization means, stabilizing the position of the implant 1 to the glenoid bone G, subsequently threads or strands are inserted along the holes 6 and the channels Cg obtained in the glenoid bone G in order to fix and stabilize the implant 1 to the glenoid bone G. In particular, the threads or strands 7 are then knotted on one or both sides of the glenoid bone G.

In conclusion, the hybrid implant according to the invention, in the embodiments described, enables to solve different problems tied to various weak points of everything that has been described up to now in the scientific literature regarding the use of bone grafts.

The implant according to the invention can be implanted by minimally invasive arthroscopy with maximum safety because the second element made of biocompatible material is in the wedge portion of the implant, thus reducing the risk of breakage of the bone portion during implantation.

Furthermore, the implant according to the invention intrinsically has a greater stabilizing capacity than prior art grafts, since a portion of the first element of the implant is wedged in the slotted glenoid bone, reinforced by the second element made of rigid osteoinductive material.

Moreover, the implant according to the invention, with respect to what has been described for correcting the glenoid defect, has the following advantages:
it can be inserted by arthroscopy;
it reduces surgery times;
it improves the characteristics of synthesis thanks to the rigid portion, which, by penetrating effectively into the glenoid bone, reinforces the anchorage of the implant to the glenoid cavity;
it eliminates the possibility of rotation and microinstability of the implant.

Furthermore, the implant according to the invention increases graft-glenoid cavity synthesis compared to the old systems, leaving the function of temporary protection of the underlying structure and of transport up to the button resting on the bone part.

In the foregoing, the preferred embodiments have been described and variants of the present invention have been suggested, but it is to be understood that a person skilled in the art may introduce modifications and changes without going outside the scope of protection, as defined by the appended claims.

What is claimed is:

1. Implant (1) for the correction of gleno-humeral instability, in particular for the correction of a glenoid defect (D_{g}) of the glenoid (G) of a patient, said implant (1) having a substantially flat portion (3), said substantially flat portion (3) being suitable, when said implant (1) is in use, for being placed at the site of the glenoid defect (D_{g}) in contact with the external bone portion of the glenoid (G) of a patient, said implant (1) being characterized in that at least said flat portion (3) is made of heterologous deproteinized bone material.

2. Implant (1) according to claim 1, wherein it has at least a first hole (6) in the flat portion (3) suitable for allowing the passage of at least a thread or strand (7) for the stabilization of the implant (1), preferably it has two holes (6).

3. Implant (1) according to claim 1, wherein the cortical part of the bone is arranged in correspondence of a first outer side of the flat portion (3) of the implant (1) and the spongier part of the bone is arranged in correspondence of a second inner side of the flat portion (3), opposite to the first side.

4. Implant (1) according to claim 3, wherein it has at least a first through hole (6) passing through said first outer side and send second inner side of the flat portion (3).

5. Implant (1) according to claim 1, wherein said flat portion (3) has a plurality of holes (10, 6) suitable for allowing the insertion of stabilization means (5; 7, 8; 7; 13, 8) for facilitating the stabilization of the implant (1) in the glenoid (G).

6. Implant (1) according to claim 1, wherein it has a substantially J-shape with a wedge portion (2) substantially transversal to said substantially flat portion (3), said wedge portion (2) being suitable for being inserted into the bone channel (C_{g}) obtained in the glenoid (G) of a patient, in that said wedge portion (2) is made of a biocompatible rigid material is coupled to said flat portion (3).

7. Implant (1) according to claim 6, wherein said flat portion (3) and said wedge portion (2) are integral with each other and are made of heterologous deproteinized bone material.

8. Implant (1) according to claim 6, wherein it has at least a second hole (12) obtained in the wedge portion (2) suitable for allowing the passage of at least a thread or strand (13).

9. Implant (1) according to claim 6, wherein the wedge portion (2) is coupled to the flat portion (3) by means of coupling means (9).

10. Implant (1) according to claim 9, wherein said wedge portion (2) is made of a porous or non-porous biocompatible material, or a biocompatible metal such as titanium or tantalum.

11. Implant (1) according to claim 9, wherein the flat portion (3) provides a plurality of holes (10), in that the coupling means are a plurality of pins (9) in said wedge portion (2) suitable for being inserted in the respective holes (10) of said flat portion (3).

12. Implant (1) according to claim 6, wherein the wedge portion (2) has a plurality of holes (11) that make up a rough surface, so as to facilitate the grasp of the implant (1) on the bone channel (C_{g}).

13. Implant (1) according to claim 10, wherein the wedge portion (2) of the implant (1) has a rough surface obtained by knurling, so as to facilitate the grasp of the implant (1) on the bone channel (C_{g}).

14. Implant (1) according to claim 6, wherein the wedge portion (2) of the implant (1) is substantially curved so as to more effectively grasp the bone channel (C_{g}) wherein it is inserted.

15. Kit comprising the implant (1) according to claim 1, and stabilization means (5; 7; 7, 8; 12, 8) for facilitating the stabilization of the implant (1) in the glenoid (G).

16. Kit according to claim 15, wherein said stabilization means comprise at least a thread or strand (7; 13).

17. Kit according to claim 16, wherein said stabilization means further comprise at least one button (8) suitable for coupling with said at least one thread (7; 13).

18. Kit comprising the implant (1) according to claim 6, and stabilization means (5; 7; 7, 8; 12, 8) for facilitating the stabilization of the implant (1) in the glenoid (G), wherein said stabilization means comprise at least a second element (5), said second element being a sheath (5) that, when it is coupled to the implant (1), at least partially overlaps the wedge portion (2).

19. Kit according to claim 18, wherein said sheath (5) has a mesh or grid that forms a rough surface, so as to facilitate the grasp of the implant (1) on the bone channel (C_{g}).
